(19) United States
(12) Patent Application Publication
(10) Pub. No.: US 2016/0356757 A1
(43) Pub. Date: Dec. 8, 2016

(54) METHOD AND APPARATUS FOR CONTINUOUSLY COLLECTING DEPOSITS FROM INDUSTRIAL PROCESS FLUIDS FOR ONLINE-MONTORING AND FOR RECORD KEEPING

(71) Applicant: Solenis Technologies, L.P., Schaffhausen (CH)

(72) Inventors: Fushan Zhang, Hockessin, DE (US); Terry Lynn Bliss, Wilmington, DE (US); Sarah D. Garchinsky, Newark, DE (US)

(21) Appl. No.: 14/729,603

(22) Filed: Jun. 3, 2015

(51) Int. Cl. G01N 33/18

(52) U.S. Cl. CPC G01N 33/1826

(57) ABSTRACT

The current apparatus and method relate to a process for determining the amount and type of organic contaminants in industrial process fluids. The current process enables an industrial plant to quickly analyze the process fluid and thus control the amount of deposition that takes place on the surfaces of equipment in contact with the process fluid.

### BACKGROUND OF INVENTION

The current apparatus and method relate to a process for determining the amount and type of organic contaminants in industrial process fluids. The current process enables an industrial plant to quickly analyze the process fluid and thus control the amount of deposition that takes place on the surfaces of equipment in contact with the process fluid.

The deposition of organic contaminants (i.e., pitch and stickies) on equipment surfaces in industrial water processes, such as the petroleum industry, food processing industry, industrial water treatments, and the pulp and papermaking industry, are well known to be detrimental to both product quality and the efficiency of the industrial processes. For example, some contaminating components occur naturally in wood and are released during various pulping and papermaking processes. Two specific manifestations of this problem are referred to as pitch (primarily natural resins) and stickies (adhesives or coatings from recycled paper). Pitch and stickies have the potential to cause problems with deposition, quality of the paper, and efficiency in the papermaking process.

When organic contaminants, such as pitch and stickies, deposit on surfaces in papermaking, quality and operating efficiency of a pulp or paper mill may be impacted or reduced. Organic contaminants can deposit on process equipment in papermaking systems resulting in operational difficulties in the systems. When organic contaminants deposit on consistency regulators and other instrument probes, these components can be rendered unreliable or useless. Deposits on screens can reduce throughput and upset operation of the system. This deposition can occur not only on metal surfaces in the system, but also on plastic and synthetic surfaces, such as machine wires, felts, foils, Uhle boxes and headbox components.

Historically, the subsets of the organic deposit problems, “pitch” and “stickies”, have manifested themselves separately, and differently, and have been treated separately and distinctly. From a physical standpoint, “pitch” deposits usually have been formed from microscopic particles of adhesive material (natural or man-made) in the stock that accumulate on papermaking or pulping equipment. These deposits readily can be found on stock chest walls, paper machine foils, Uhle boxes, paper machine wires, wet press felts, dryer felts, dryer cans, and calendar stacks. The difficulties related to these deposits include direct interference with the efficiency of the contaminated surface, leading to reduced production, as well as holes, dirt, and other sheet defects that reduce the quality and usefulness of the paper for operations that follow, like coating, converting or printing.

Pitch causes many issues in process plants and generally is used to describe deposits composed of organic constituents that may originate from natural wood resins, their salts, as well as coating binders, sizing agents, and defoaming chemicals which may be found in the pulp. In addition, pitch frequently contains inorganic components, such as calcium carbonate, talc, clays, titanium and related materials.

“Stickies” is a term that has been increasingly used in process plants to describe deposits that occur in systems using recycled fibers. These deposits often contain the same materials found in “pitch” and also may contain adhesives, hot melts, waxes, binders, and inks.

From a physical standpoint, “stickies” usually consist of particles of visible or nearly visible size in the stock that originate from recycled fiber. These deposits tend to accumulate on many of the same surfaces on which “pitch” can be found and cause many of the same difficulties that “pitch” can cause. The most severe “stickies” related deposits, however, tend to be found on papermaking machine wires, wet felts, dryer felts and dryer cans.

Methods of preventing the build-up of deposits on the pulp and paper mill equipment and surfaces are of great importance to the industry. The paper machines could be shut down for cleaning, but ceasing operation for cleaning is undesirable because of the consequential loss of productivity. Deposits also can result in poor product quality, which occurs when deposits break off and become incorporated in the sheet. Preventing deposition thus is greatly preferred where it can be practiced effectively.

In the past, stickies deposits and pitch deposits more typically have manifested themselves in different systems. This was true because mills usually used only virgin fiber or only recycled fiber, and did not mix these furnish slurries together. Often very different treatment chemicals and strategies were used to control these separate problems.

Current trends are for increased use of recycled fiber in papermaking systems. This is resulting in a co-occurrence of stickies and pitch problems in a given mill. In addition, with increased recycling of fibers, the trend towards “microstickies”, defined as stickies with a diameter less than 150 μm, is a growing concern. Microstickies, because of their small size and large surface area, present a greater tendency to deposit and/or agglomerate.

In order to determine the contaminant content in a pulp, methods that measure the deposition of organic contaminants on a specific substrate by gravimetric analysis have been used. U.S. Pat. No. 6,090,905, teaches a method wherein the weight differential of packaging foam, placed in stainless steel baffles, before and after exposure to pulp slurry is utilized to estimate the content of deposited stickies. European Pat. No. EP 0 922 475A1 discloses a device that accumulates deposits under a shear field brought about by a rotating disc.

A variation to the gravimetric methods to quantify deposition is the use of sensors that respond to the weight of the deposit. U.S. Pat. No. 5,646,338 teaches an apparatus that relates the amount of lateral deflection about a pivot of a cantilever probe to the build-up of deposit on the projection portion of said probe. U.S. Pat. Appl. Pub. No. 2006/0281191 A1 teaches the use of a quartz crystal microbalance whose vibration frequency and amplitude are affected by the formation of deposits on the exposed side of the crystal.

A drawback with gravimetric methods of measuring deposition quantity is that the actual measurement has a high potential for variability because of the small weight of deposit on the substrate. Sensors can also be problematic when they are introduced into high shear environments or where there are mechanical vibrations in the fluid. Consequently, these methods may not be able to characterize the efficacy of a deposition treatment program.

Methods to specifically quantify stickies in pulp are summarized by J. Dyer, “A Summary of Stickies Quantification Methods,” Progress in Paper Recycling, pp. 44-51 (1997, August). These methods include image analysis techniques, such as that employed in conjunction with the Pulmac MasterScreen (Pulmac International, Montpelier, Vt., USA) low consistency screening device designed to mechanically separate fibers from contaminants including stickies and shives. R. Blanco, et al., “New System to Predict Deposits due to DCM Destabilization in Paper Mills,” Pulp & Paper Canada, 101(9), pp. 40-43 (2000), discloses a variation of the equipment disclosed in European Patent No. EP 0 922 475A1 employing image analysis techniques. K. Cathie, et al., “Understanding the Fundamental Factors Influencing Stickies Formation and Deposition,” Pulp & Paper Canada, 93(12) (1992), pp. 157-160, discloses a method wherein stickies deposition on forming wires is quantified by image analysis.

U.S. Pat. No. 8,160,305 discloses an apparatus for in-line particulate contamination collection for the measuring depositability of particulate contaminants in pulp and paper systems. However, the apparatus requires a sampling chamber with an inlet and an outlet into which is directed a stream of a pulp slurry or process water, and has no mechanism of controllable deposition rate by adjusting temperature of substrate surface or spraying a stream of pulp slurry or process water directly on the substrate surface.

WO Publication No. 2011/072396 A1 and M. Richard, et al., “A New Online Image Analyzer for Macrocontaminants in Recycled Pulp,” TAPPI Journal, 11(2) (2012), pp. 19-28, discloses an image analysis-based online macrocontaminant analyzer. It only characterizes macrocontaminants larger than 200 μm and requires a mechanical separation of macrocontaminants from fibers in pulp slurry.

U.S. Patent Publication No. 2012/0258547 discloses a device and method to perform real-line macrostickies and/or any visible hydrophobic particle analysis in an aqueous medium. The technique is based on fluorescence image analysis to identify and measure sticky particles, and, requires addition of a hydrophobic dye into the aqueous pulp slurry.

Suvajit Das, “Real-time in-line measurement of stickies in recycled pulp”, 2014 TAPPI PEERS Conference, discloses a stickies sensor using an inline artificial-intelligence-based system built on machine vision and pattern recognition.

Some image analysis techniques do discriminate between different types of contaminants to quantify those which specifically result in deposition. However, they typically are not capable of quantifying microstickies. Improved methods and apparatus for collecting particulate contaminants, diagnosing stickies and pitch formation and evaluating effectiveness of prevention treatments continue to be sought.

In order to deal with the problems caused by the organic contaminants in a papermaking or pulping system, it is desired to have an apparatus and method for real-time monitoring that can rapidly measure depositable contaminants in industrial operations such as papermaking and pulping processes, so that an operator of the processing plant can timely predict likelihood of contaminant deposition, and take the appropriate measures to prevent or control the deposition.

### SUMMARY

The present process relates to a method of sampling and analyzing process fluids for determination of deposits in industrial processes and for online monitoring and record keeping. A continuous substrate having a portion of its surface designated a deposit collection area is advanced through a deposit collection zone, wherein a stream of the process fluid is brought into contact with a substrate surface collection area as the substrate is advanced through the deposit collection zone. As the substrate is advanced, there is an optional rinse step wherein the substrate is rinsed with water. The substrate is then advanced through another area designated as a deposit inspection zone, wherein the deposits that had been collected on the substrate can be analyzed using an online analysis and/or monitoring technique. The substrate is then collected for disposal or, for optionally saving the substrate for further analysis. The substrate can be advanced continuously or intermittently throughout the process.

The present process also relates to an apparatus that samples process fluids for deposits from industrial process fluids for online monitoring and record keeping. A continuous substrate is provided in the form of a tape wound on a tape supply roll. The substrate, which has one side designated as the deposit collection area, is advanced, continuously or intermittently, and in contact with a support block to a deposit collection zone. The support block guides and supports the substrate without the use of pulleys or rollers, which is problematic in some industrial fluids. A nozzle directs a stream of process fluid into direct or indirect contact with the deposit collection area of the substrate. The excess fluid is collected in a flow tray or tank and discarded or recirculated. Flow tank or tray are used interchangeably herein. The support block, substrate and spray nozzle can be positioned above the flow tank and above the level of the process fluid in the flow tank or within the flow tank below the level of the process fluid in the flow tank. The substrate can be advanced through a rinse cycle and the substrate inspected for deposits in an area designated as the deposit inspection zone. In this area the deposits, in the form of pitch or stickies that were collected on the substrate in the deposit collection area, are analyzed using at least one quantitative and/or qualitative analysis method. The substrate tape can be collected on a take-up or collecting roll after being analyzed for deposits and can be kept for further analysis or discarded. Advancement of the tape through the deposition collection and inspection zones can be in a continuous or intermittent mode. The term flow tank or flow tray is used interchangeably herein.

The apparatus can be optionally equipped with a temperature controlling device to control the temperature of substrate surface through a temperature controlled support block. The process fluids may also be temperature controlled. This may better simulate the real-time run conditions of the process fluids.

### BRIEF DESCRIPTION OF THE DRAWINGS

FIG. 1, depicts one variation of the current apparatus.

FIG. 2, depicts a second variation of the current apparatus.

FIG. 3, is a graph representing the results of the percent area occupied by the deposited particles in each acquired image (% AOI) and particle counts representing the total deposited particles identified in each acquired image

### DETAILED DESCRIPTION

In one aspect of the current invention, there is an apparatus and method for determining contaminants in the form of deposits that are found in industrial process fluids.

In another aspect, there is an apparatus for sampling industrial process fluids for determining deposits found in the process fluid wherein there is a supply means for delivering a continuous substrate having a portion of its surface designated a deposit collection area and a collecting means for collecting the continuous substrate; a support block to guide the continuous substrate from the supply means to the collecting means;

a deposit collection zone wherein deposits present in the process water are deposited on the deposit collection area of the substrate; an optional rinse bath or spray; a deposit inspection zone, wherein the deposits collected on the substrate are analyzed using at least one online analysis method.

In some aspects, the apparatus referred to directly above can have an optional rinse bath or spray.

In still other aspects, the apparatus as described above can have a support block or guide that can be temperature controlled.

In another aspect, there is a method for sampling industrial process fluids for determining deposits comprising: 1) providing a continuous substrate having a portion of its surface designated a deposit collection area; 2) advancing the substrate, continuously or intermittently, while in contact with a support block to a deposit collection zone; 3) exposing the surface designated as the deposit collection area to the industrial process fluid to collect deposits; (4) optionally the substrate can be rinsed in place or advanced through a rinse bath or spray; (5) advancing the substrate through a deposit inspection zone; (6) analyzing the deposits collected on the substrate at the deposit inspection zone using at least one online analysis method; and (7) collecting the substrate.

In another aspect of the current process, prior to the substrate being advanced to the deposit inspection zone as described in the aspect above, there can be a rinse stage after the substrate goes through the deposit collection zone. The substrate may be rinsed in the deposit collection zone by replacing the process fluid with a rinse solution.

The industrial process fluids of the current process, refers to the fluids used in industries such as, the petroleum industry, food processing industry, industrial water treatments, and the pulp and papermaking industry.

In other aspects, the continuous substrate on which the deposit may be collected include, but are not limited to, metals or metal foil that represent machine surfaces; plastics or plastic films that represent forming wires and felts; surfaces of any generally liquid impermeable material that may become coated with components of organic contaminants from recycle fiber sources; and surfaces of any generally liquid impermeable material which may become coated with components of organic contaminants from virgin fiber surfaces. The latter two types of substrate surfaces simulate the growth of an already formed deposit in the presence of particulate.

Additional examples of a suitable substrate includes plastic or plastic film substrates include, but are not limited to, polypropylene, polyethylene, polyvinyl chloride (PVC) or polyvinylidene chloride (PVDC), or polyesters, such as polyethylene napthate (PEN) or Polyethylene Terephthalate (PET). The substrate can be transparent plastic films which allow the passage of visible light. These plastic film substrates are generally liquid impermeable and may carry a coating thereon. Suitable metal substrates include, but are not limited to, stainless steel or carbon steel. These metal substrates are generally liquid impermeable and may carry a coating thereon.

In a further aspect of the invention the suitable substrate can comprise a layer or film of similar or the same composition as the particulate component or other organic deposits. For example, a transparent plastic substrate coated with a non-water soluble adhesive, such as Scotch® brand transparent adhesive tape manufactured by 3M (St. Paul, Minn., USA), is a suitable substrate. The substrate can be a substrate Scotch® Sure Start.

The deposit collection area of the substrate encompasses the area of the substrate that comes into direct contact with the stream or spray or circulation of the process fluid.

The deposit collection zone, is the stage of the process wherein the substrate is placed in a position to come into contact with the process fluid being measured for contaminants.

In some aspects of the current process, there can be a rinse stage subsequent to the deposit collection zone. At this stage the substrate is advanced through, for example, a rinse bath and/or a spray shower in which a stream of rinse fluid or water is directed onto the deposition area of the substrate. The rinse stage washes loosely attached particulate from the surface of the substrate. The substrate can be advanced continuously or intermittently through the current process with the process fluid being replaced by a rinse solution after the deposition stage.

After the substrate advances through the deposit collection zone and optional rinse step, the substrate is analyzed for deposits. Various analysis tools can be utilized at this zone to online inspect or monitor the deposits collected on the substrate. For example, online analysis techniques can be selected from UV-Visible spectroscopy, Fourier transform infrared spectroscopy (FTIR), Raman spectroscopy, X-ray Fluorescence Spectroscopy (XRF), imaging analysis, or any combination of them.

In other aspects of the current process, the collected substrate containing the deposits can either be saved as material records for future reference or the substrate can be re-analyzed with various analysis techniques such as UV-Visible spectroscopy, Fourier transform infrared spectroscopy (FTIR), Raman spectroscopy, X-ray Fluorescence Spectroscopy (XRF), imaging analysis, or any combination thereof.

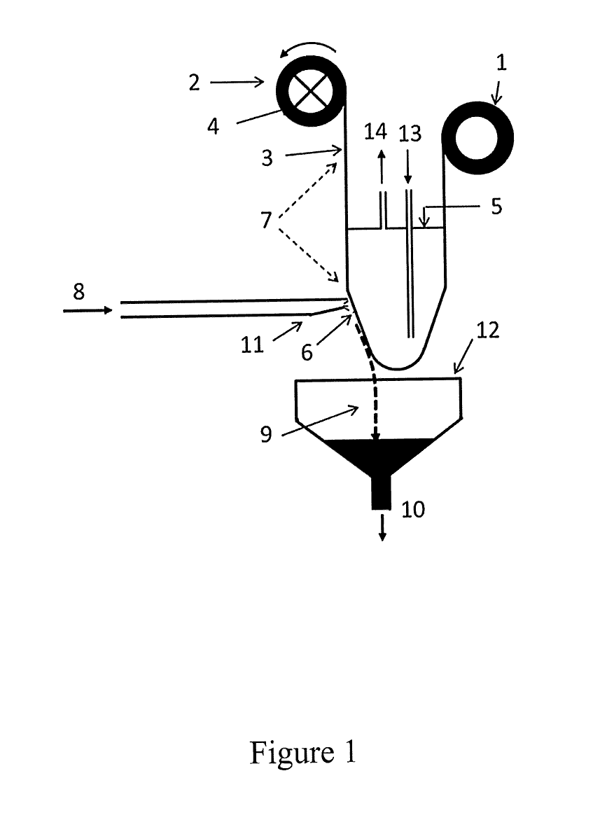

In another aspect, referring to FIG. 1, the substrate is in the form of a roll of tape. The substrate tape (3) is unwound from its supply roll (1) and brought into contact with a support block (5), with a portion of the surface of the substrate designated as a deposit collection area, into a deposit collection zone where the process fluid comes into contact with the deposit collection area of the substrate (6). The substrate tape continues to be advanced, continuously or intermittently, to a deposit inspection zone (7), which can be located above the collection zone (6). Finally, the substrate tape is collected on a take-up roll (2). The substrate tape can be collected continuously or intermittently with the take-up roll being driven by a driving motor (4) or alternatively by a friction roll.

As the substrate advances through the deposit collection zone (6), a stream of process fluid (8) from an industrial process is continuously sprayed onto the substrate tape (3). In some embodiments, this is accomplished via a spray nozzle (11). After spraying a stream of process fluid onto the substrate, the process fluid (9) is collected into a flow tray (12), thereafter, discharging the stream of process fluid (10) either to sewage or back to the industrial process.

In another aspect, referring to FIG. 1, when the process is run continuously, the deposit collection zone (6) of the current apparatus can be either located in the air or submerged in the process fluid, and in the side either with the tape take-up roll (2) or with the tape supply roll (1). When the deposit collection zone (6) is located in the position illustrated in FIG. 1, on the same side of the tape take-up roll (2), a faster response time can be expected at a given tape speed.

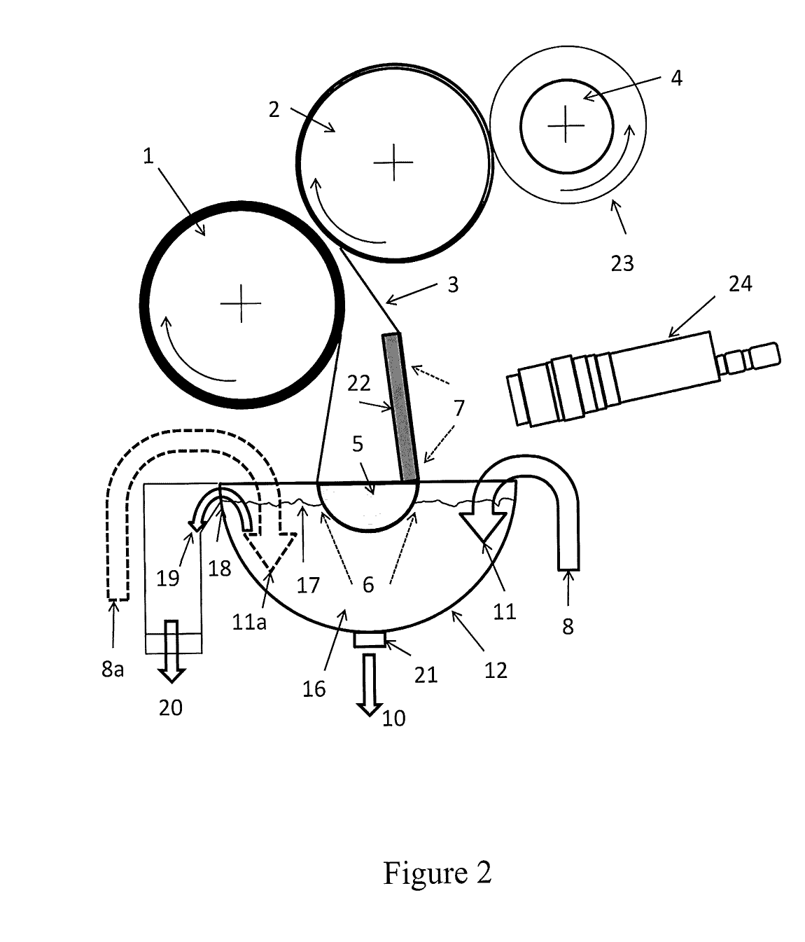

In another aspect of the current apparatus, referring to FIG. 2, the substrate tape (3) can be advanced, continuously or intermittently, while in contact with a support block (5). The support block (5) is positioned within the flow tank (12) and flow tank filled with process fluid (8 and 8a) either through a spray nozzle (11 and 11a) or another separate inlet (not depicted). The process fluid (16) and process fluid flow level (17) in the flow tank (12) is such that the support block (5) and substrate tape (3) is below the level in the process fluid. The flow tank drain (21) can have a shut-off valve to prevent the process fluid (10) from immediately draining from the flow tank (12). The substrate tape is then advanced, continuously or intermittently, through the deposit inspection zone (7), which may contain an LED pad (22), for analysis by an analytical device such as a camera (24). The tape is collected on a take-up roll to be discarded or kept for future reference.

In some aspects of the apparatus, referring to FIG. 2, the spray nozzle (11 and 11a) is below the level of the process fluid (17) in the flow tank (12). The process fluid in the flow tank can be re-circulated through the spray nozzle (1 and 11a) causing turbulence and contact of the process fluid with the deposit collection area (6) of the substrate (3) or fresh process fluid from another source can be used.

In yet other aspects of the apparatus, referring to FIG. 2, in addition to a flow tank drain (21) the apparatus can have an overflow weir (18). This may be used to maintain a certain level of process fluid (17) in the flow tank (12) as the overflow weir would provide an outlet for overflow process fluid (19) to be discharged (20).

In some aspects of the current process, the contact time of the process fluid (16) with the deposit collection area of the substrate can be from a few seconds to several hours. Contact time can be for about 60 seconds to about 1 hour and may be for 5 minutes to about 30 minutes. Optimum contact time is largely dependent upon the type of process fluid being analyzed.

In another aspect of the current apparatus, referring to FIG. 2, the take-up roll (2) can have a drive motor (4) attached to the take-up roll or the drive motor can drive a rider roll (23).

In some aspects of the apparatus, the supply roll (1), support block (5) and take-up roll (2) can be raised or lowered to a desired position above or below the level of the process fluid (17) in the flow tank (12). Raising the rolls and support block can also help facilitate changing the substrate on the rolls. In other aspects, the flow tank can be lowered.

In another aspect, the analytical method used in the inspection zone, can detect depositable contaminant particles in a wide range of particle size, including deposits that have a particle size of 5 micron or less.

In one aspect of the current process, analysis of the deposits can be done automatically and continuously for a period of time of from several hours to several months, without human intervention.

In yet another aspect, the current online analysis and real-time monitoring techniques of the present method can effectively preserve contaminant particles that are collected during a papermaking or pulping process as material evidence for record keeping.

The choice of the online analysis methods depends on the nature of the deposits and the objectives of the inspection.

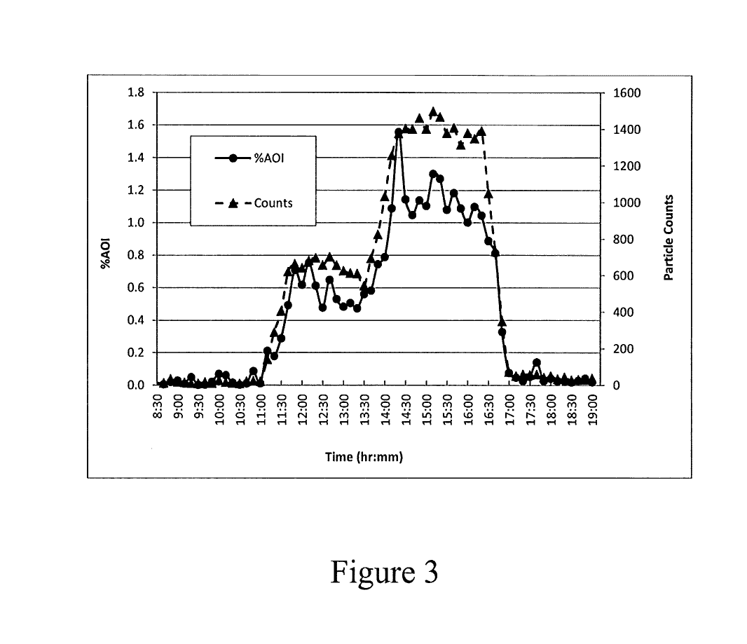

In other aspects of the method as described above, referring to FIGS. 1 and 3, the temperature of the substrate surface (3) can optionally be adjusted as the substrate tape is advanced over the support block (5) into the deposit collection zone (6). Adjusting the temperature of the substrate surface can help to enhance the efficiency of deposit collection on the substrate surface as it is possible to simulate actual operating temperature of other industrial processes. Temperature of the substrate surface can also be adjusted to maintain the temperature stability during the collection of deposits on the substrate. In addition to the temperature of the substrate being controlled, the temperature of the process fluid can also be controlled.

In one aspect, the support block as described herein can be heated by a cartridge heater control means.

By controlling the temperature of the substrate and/or process fluid it was found that deposition of contaminants onto the substrate was enhanced.

In some aspects of the current process, the temperature of the substrate can be controlled by circulating a heat transfer fluid through the substrate support block (5) via an inlet (13) and outlet (14) port(s). The heat transfer fluid is preheated or cooled by a separate temperature controlling device, such as a thermal circulator. The fluid can be selected from water, antifreeze, water-based coolants or various oils such as mineral oils, castor oils, and silicone oils. Choice of the temperature of substrate surface will depend on the nature of the deposits to be collected, the normal process temperature of the industrial fluid, and objectives of collecting deposits. The temperature of the substrate can range from about 1° C. to about 99° C. and can range from about 20° C. to about 70° C. for water based process fluids, such as papermaking slurries. Other industrial fluids may require higher temperatures.

In another aspect, the spray pattern of the process fluid stream on the substrate surface can be, but is not limited to, a solid stream, flat spray, full cone, hollow cone or any pattern or spray nozzle used by those skilled in the art. The spray pattern can be a plate spray, which appears as a flat sheet of liquid and can be a solid stream.

In yet another aspect of the current apparatus, the spray nozzle can be a plain orifice nozzle. An orifice nozzle often produces little if any atomization, but directs the stream of fluid in certain spray patterns. The geometry of the orifice of a suitable nozzle for the current apparatus include, but are not limited to, circle, ellipses, circular segment, and rectangles, etc., as known to those skilled in the art. In addition, the opening of the orifice of the suitable nozzle can be in range of from about 1 millimeter squared (mm²) to about 60 mnm²; can be in a range of from about 5 mm² to about 50 mm²; and may be in range of from about 10 mm² to about 40 mm².

In yet another aspect of the current apparatus, in order to minimize or to eliminate the interference of splash caused by spraying the process fluid onto the substrate surface, a splash guard above the spray nozzle can be incorporated.

In one aspect of the current process, the spray distance, when used with the spray patterns and spray nozzles described above and is defined as the closest distance from the orifice of the spray nozzle to the substrate surface, can be from about 0.5 mm to about 50 mm; can be from about 1 mm to about 25 mm; and may be from about 2 mm to about 5 mm.

In another aspect of the current process, a suitable flow rate, using the spray patterns, nozzles and distances disclosed in the above paragraphs, of the industrial process fluid can be from about 10 milliliter per minute (ml/minute) to about 20,000 ml/minute; can be from 100 ml/minute to about 10,000 ml/minute; and may be from about 200 ml/minute to about 6,000 ml/minute.

Some suitable substrates are taught in U.S. Pat. No. 8,160,305, incorporated herein by reference. Choice of the substrate will mainly depend on nature of deposits to be collected and the methods that are employed to inspect the deposits. For example, when the invented apparatus is employed to collect stickies contaminant particles from pulp slurry or the process water in paper recycling mills and an image processing method is employed to inspect the collected deposit particles, the substrate can be a transparent plastic substrate coated with a non-water soluble adhesive, such as Scotch® Shipping Packaging Tape, Scotch® Moving Storage Packaging Tape, or Scotch® Sure Start Shipping Packaging Tape, manufactured by 3M (St. Paul, Minn., USA).

In all aspects of the current process discussed above, it is understood that the substrate can travel in a continuous or an intermittent mode through the deposit collection zone. Although the optimum speed of the substrate through the deposit collection zone is dependent upon the type of process fluid being analyzed, the moving speed of the substrate can be in a range of from about 0.1 mm/minute to about 200 mm/minute; can be in the range of from about 1 mm/minute to about 100 mm/minute; and can be in range of about 2 mm/minute to about 50 mm/minute.

In some aspects of the current process, the apparatus collects deposits from various process fluids from any industry that often has issues of deposits on surfaces of process equipment, such as in the petroleum industry, food processing industry, industrial water treatments, and the pulp and papermaking industry. When the current apparatus is used in the pulp and papermaking industry, organic and inorganic contaminants can be measured and controlled. Organic deposits can include pitch, white pitch and stickies. One of the most common biomass deposits in pulp and papermaking processes are biofilm. Other contaminants can include various inorganic scales, organic contaminant deposits and biomass deposits from various pulping and papermaking processes. Inorganic deposits include various scales, such as calcium carbonate (CaCO₃.H₂O), calcium oxalate (CaC₂O₄), barium sulfate (BaSO₄), calcium sulfate (CaSO₄), aluminum hydroxide (Al(OH)₃), as found in the industries being served.

In some aspects, the current apparatus can be used in chemical pulping processes such as in a kraft or sulfite process and various mechanic pulping processes such as stone groundwood pulping (SGW), pressure groundwood pulping (PGW), thermo-mechanical pulping (TMP), or chemi-thermo-mechanical pulping (CTMP), etc.

The following examples are provided to illustrate the apparatus and process for determining deposits in process fluids. One skilled in the art will appreciate that although specific features and conditions are outlined in the following examples, these features and conditions are not a limitation on the present teachings.

### EXAMPLES

### Example 1

### Spray Configuration of the Apparatus

An apparatus, configured similar to the apparatus depicted FIG. 1, was built and tested as follows: A Scotch® Sure Start Shipping Packaging Tape was used as the continuous substrate (3) and mounted on the tape supply roll (1) of the device. A motor was installed on the substrate tape take-up roll (4) and the substrate tape (3) was unwound from its supply roll (1), passed under a shaped support block (5) having a rounded bottom and the substrate advanced through the deposit collection zone and re-wound at the tape take-up roll (2). The substrate tape was advanced at a speed of 246 millimeter per hour. The substrate deposition area faced outward as the substrate advances over the shaped support block. In this example the deposit collection zone (6) of the apparatus was located on the same side as the tape take-up roll (2). The spray nozzle (11) was a PVC tube with 12.5 mm inner diameter. A deposit inspection zone (7) was located just prior to the substrate tape take-up roll and equipped with a Keyence Machine Vision XG7702 image system.

The process fluid being tested was a pulp slurry. Thirty 30 liters (L) of 0.5% bleached Kraft hardwood pulp slurry was provided in a 55 liter storage tank, which was further equipped with an overhead mixer operating at 500 revolutions-per-minute (rpm) and a fluid pump. During the test, the pulp slurry was continuously pumped from the storage tank to the nozzle and sprayed onto the substrate tape collection area, and the pulp slurry returned to the tank through a recirculation line. The image analysis system automatically acquired images of the substrate tape that was passing the deposit inspection zone and results of image analysis were gathered at a rate of every 10 minutes per image. The camera of the image system was set to a focal distance of 100 millimeters (mm) and the field of view of 40 millimeters (mm) by 40 mm. Image analysis determines the % Area of Interest (% AOI), which is the percent area occupied by the deposited particles in each acquired image, and the Particle Counts, which is the total number of deposited particles identified in each acquired image. Following are the events during the test:

A: At 8:30 AM, started the test by circulating 30 liters of 0.5% bleached kraft hardwood pulp in a 55 liter agitated storage tank.

B: At 10:30 AM, 2 liters of 0.5% solids slurry formed from sorted office waste (“SOW”) from a recycle paper mill was added to the pulp storage tank, equivalent to adding 6.3% SOW pulp into the system, based on dry weight. The SOW slurry contained a high concentration of“stickies”.

C: At 1:00 pm, 3 liters of the 0.5% SOW was added again to the pulp storage tank, equivalent to adding a sum total of 14.3% SOW pulp to the system (including the first addition), based on dry weight.

D: At 3:50 pm, 0.6 g of DeTac DC779F detackifier (14.5% solid, obtained from Solenis LLC, Wilmington, Del.) was added to the pulp storage tank, equivalent to 1 pound of detackifier product per ton of paper, based on dry weight.

E: At 7:00 pm, the test was ended.

TABLE 1
% AOI and Particle Counts

| | Time | % AOI | Counts | Time | % AOI | Counts |
|---|---|---|---|---|---|---|
| 8:20:52 | 8:30 | 0.0063 | 13 | 13:50 | 0.7456 | 825 |
| 8:30:52 | 8:40 | 0.0056 | 12 | 14:00 | 0.7888 | 1032 |
| 8:40:52 | 8:50 | 0.0201 | 34 | 14:10 | 1.0885 | 1255 |
| 8:50:52 | 9:00 | 0.0279 | 19 | 14:20 | 1.5581 | 1376 |
| 9:00:52 | 9:10 | 0.0105 | 15 | 14:30 | 1.1444 | 1403 |
| 9:10:52 | 9:20 | 0.0489 | 11 | 14:40 | 1.0486 | 1401 |
| 9:20:52 | 9:30 | 0.0034 | 10 | 14:50 | 1.1374 | 1461 |
| 9:30:52 | 9:40 | 0.0037 | 17 | 15:00 | 1.1041 | 1401 |
| 9:40:52 | 9:50 | 0.021 | 11 | 15:10 | 1.3 | 1497 |
| 9:50:52 | 10:00 | 0.0691 | 28 | 15:20 | 1.2711 | 1466 |
| 10:00:52 | 10:10 | 0.0616 | 16 | 15:30 | 1.0805 | 1378 |
| 10:10:52 | 10:20 | 0.0151 | 12 | 15:40 | 1.1827 | 1407 |
| 10:20:52 | 10:30 | 0.0042 | 9 | 15:50 | 1.0885 | 1314 |
| 10:30:52 | 10:40 | 0.0107 | 22 | 16:00 | 1.0027 | 1378 |
| 10:40:52 | 10:50 | 0.0872 | 24 | 16:10 | 1.0981 | 1348 |
| 10:50:52 | 11:00 | 0.0122 | 22 | 16:20 | 1.0453 | 1390 |
| 11:00:52 | 11:10 | 0.2118 | 141 | 16:30 | 0.8891 | 1049 |
| 11:10:52 | 11:20 | 0.1798 | 289 | 16:40 | 0.813 | 732 |
| 11:20:52 | 11:30 | 0.2887 | 405 | 16:50 | 0.3287 | 349 |
| 11:30:52 | 11:40 | 0.4932 | 622 | 17:00 | 0.0758 | 72 |
| 11:40:52 | 11:50 | 0.709 | 663 | 17:10 | 0.0481 | 50 |
| 11:50:52 | 12:00 | 0.6188 | 642 | 17:20 | 0.0275 | 60 |
| 12:00:52 | 12:10 | 0.7545 | 681 | 17:30 | 0.0524 | 56 |
| 12:10:52 | 12:20 | 0.6135 | 696 | 17:40 | 0.1417 | 59 |
| 12:20:52 | 12:30 | 0.4774 | 658 | 17:50 | 0.0274 | 42 |
| 12:30:52 | 12:40 | 0.649 | 702 | 18:00 | 0.0406 | 50 |
| 12:40:52 | 12:50 | 0.5307 | 657 | 18:10 | 0.0238 | 34 |
| 12:50:52 | 13:00 | 0.4835 | 626 | 18:20 | 0.0245 | 43 |
| 13:00:52 | 13:10 | 0.5066 | 614 | 18:30 | 0.0192 | 27 |
| 13:10:52 | 13:20 | 0.4737 | 611 | 18:40 | 0.028 | 30 |
| 13:20:52 | 13:30 | 0.5611 | 544 | 18:50 | 0.0414 | 30 |
| 13:30:52 | 13:40 | 0.5824 | 694 | 19:00 | 0.0202 | 38 |

The resulting % AOI and particle counts obtained by the Keyence image system during the test can be found in Table 1 and displayed in FIG. 3. As seen, the invented apparatus effectively detected the contaminants deposited from the pulp slurry at two concentration levels, and also showed a clear response as the DcTac DC 779F detackifier from Solenis LLC, Wilmington, Del., which effectively detackified the surface of the stickie particles.

### Example 2

### Heated Support Block

The process fluid being tested was a pulp slurry. Thirty liters (L) of 1.5% consistency pulp slurry (20% SOW/80% bleached Kraft hardwood pulp) was prepared in a 55 liter supply tank, which was further equipped with an overhead mixer operating at 1000 rpm and a fluid pump. During the test, the pulp slurry was continuously pumped from the storage tank to a nozzle and sprayed onto the substrate at the deposit collection area. The pulp slurry returned to the supply tank through a recirculation line. The pulp stream was supplied to a continuous, slowly moving tape substrate (Scotch® Sure Start Shipping Packaging Tape), for 30 minutes. The substrate was continuously advanced at a rate of 1 cm/min. The pulp slurry was held at 23° C. in all trials. A heat transfer fluid (water) was circulated through the support block at 23° C. for three trials and at 60° C. for three trials. The image analysis system automatically acquired images of the substrate tape that was passing through the deposit inspection zone. Images were gathered at a rate of 1 image per minute for analysis of % AOI and particle count. Results are listed in Table 2 and Table 3.

TABLE 2
Temperature effect of heated support block on % AOI

% AOI

| | 23° C. | 60° C. |
|---|---|---|
| Trial 1 | 0.294 | 0.785 |
| Trial 2 | 0.222 | 0.779 |
| Trial 3 | 0.173 | 0.327 |
| Average | 0.230 | 0.630 |

| | |
|---|---|
| % Increase | 174.5 |

TABLE 3
Temperature effect of heated support block on Particle Count

Particle Count

| | 23° C. | 60° C. |
|---|---|---|
| Trial 1 | 336 | 876 |
| Trial 2 | 267 | 967 |
| Trial 3 | 220 | 457 |
| Average | 274 | 767 |

| | |
|---|---|
| % Increase | 179 |

The data indicates an increase in the % AOI and Particle Count when the temperature of the support block is increased to mimic the process fluid temperatures observed in a typical pulp and papermaking process. This suggests that a statistically significant number of particles can be counted in a shorter exposure time of the substrate to the process fluid if the support block is heated to mimic actual process fluid temperatures.

Each reference cited in the present application above, including books, patents, published applications, journal articles and other publications, is incorporated herein by reference in its entirety.

1. A method for sampling industrial process fluids for determining deposits comprising: 1) providing a continuous substrate having a portion of its surface designated a deposit collection area; 2) advancing the substrate, continuously or intermittently, while in contact with a support block to a deposit collection zone; 3) exposing the surface designated as the deposit collection area to the industrial process fluid to collect deposits; (4) optionally the substrate can be rinsed in place or advanced through a rinse bath or spray; (5) advancing the substrate through a deposit inspection zone; (6) analyzing the deposits collected on the substrate at the deposit inspection zone using at least one online analysis method; and (7) collecting the substrate.

2. The method according to claim 1, wherein the support block is temperature controlled.

3. The method according to claim 2, wherein the support block is heated by means of a cartridge heater.

4. The method according to claim 2, wherein the temperature of the support block is controlled by circulation of a heated or cooled heat transfer fluid.

5. The method according to claim 1, wherein the substrate is rinsed by replacing the process fluid with a rinse fluid.

6. The method according to claim 5, wherein the substrate is advanced through a spray rinse prior to the deposit inspection zone.

7. The method according to claim 1, wherein the deposit collection zone is below the level of the process fluid in the flow tank.

8. The method according to claim 1, wherein the deposit collection zone is above the level of the process fluid in the flow tank.

9. The method according to claim 1, wherein the industrial process fluids comprise the process fluids selected from the group consisting of the petroleum industry, food processing industry, industrial water treatments, and the pulp and papermaking industry.

10. The method according to claim 9, wherein the process fluid is from a pulp and papermaking mill and the process fluid is selected from the group consisting of thin stocks, thick stocks, white water, rejects and accepts of forward and reverse cleaners, rejects and accepts of screens, filtrates of thickeners and pulp washers.

11. The method according to claim 1, wherein the online analysis method is an image analysis identifying the percent area occupied by the detected deposits in each acquired image or the total counts of detected deposit particles in each acquired image, or a combination of thereof.

12. The method according to claim 1, wherein the process fluid is sprayed onto the deposit collection area of the substrate with a spray nozzle.

13. A apparatus for sampling industrial process fluids for determining deposits found in the process fluid comprising:
a supply means for delivering a continuous substrate having a portion of its surface designated a deposit collection area and a collecting means for collecting the continuous substrate;
a support block to guide the continuous substrate from the supply means to the collecting means;
a deposit collection zone wherein deposits present in the process water are deposited on the deposit collection area of the substrate;
an optional rinse bath or spray;
a deposit inspection zone, wherein the deposits collected on the substrate are analyzed using at least one online analysis method.

14. The apparatus according to claim 13, wherein the substrate surface is selected from the group consisting of metals and polymer films.

15. The apparatus according to claim 13, wherein the support block has a temperature control means.

16. The apparatus according to claim 15, wherein the support block is heated by cartridge heater control means.

17. The method according to claim 15, wherein the temperature of the support block is controlled by circulation of a heated or cooled heat transfer fluid.

18. The apparatus according to claim 14, wherein the polymer films are selected from the group consisting of polymers of polypropylene, polyethylene, polyvinyl chloride, polyvinylidene chloride and polyesters.

19. The apparatus according to claim 13, wherein the at least one online analysis method can be selected from the group consisting of UV-Visible spectroscopy, Fourier transform infrared spectroscopy, Raman spectroscopy, X-ray Fluorescence Spectroscopy, imaging analysis, and any combination thereof

20. The apparatus according to claim 13, whereas the process fluid is contacted with the substrate via a spray nozzle.

21. The apparatus according to claim 13, wherein the supply means and collecting means is in the form of a tape supply roll and tape collecting roll.
